(19) United States
(12) Patent Application Publication
(10) Pub. No.: US 2011/0238084 A1
(43) Pub. Date: Sep. 29, 2011

(54) SURGICAL INSTRUMENT

(76) Inventors: Seung Wook CHOI, Seongnam-si (KR); Jong Seok Won, Yongin-si (KR); Jae Sun Lee, Seongnam-si (KR)

(21) Appl. No.: 13/156,878

(22) Filed: Jun. 9, 2011

Related U.S. Application Data
(62) Division of application No. 13/129,334, filed on May 13, 2011.

(30) Foreign Application Priority Data
Dec. 12, 2008 (KR) 10-2008-0126415
Dec. 30, 2008 (KR) 10-2008-0136840
Dec. 30, 2008 (KR) 10-2008-0136859
Jan. 21, 2009 (KR) 10-2009-0004872

(51) Int. Cl. A61B 17/34; A61B 17/00; A61B 19/00

(52) U.S. Cl. 606/130; 606/1; 606/185

(57) ABSTRACT

A surgical instrument is discussed. The surgical instrument, which has an effector for engaging the surgical site joined to one end and a driving part for operating the effector joined to the other end, includes: a first shaft, which has one end joined with the driving part, and which extends along a first lengthwise direction; and a second shaft, which extends along a second lengthwise direction that forms a particular angle with the first shaft, and which has one end joined with the other end of the first shaft such that the second shaft is rotatable about an axis following the second lengthwise direction. Thus, it is possible to conduct surgery using several of such surgical instruments without having the instruments obstruct one another, and the surgical instrument can be made to have different usage modes according to what length it is set to.

### CROSS-REFERENCE TO RELATED APPLICATIONS

This application is a Divisional application of copending U.S. patent application Ser. No. 13/129,334, filed May 13, 2011, which is the National Phase of PCT/KR2009/007290 filed on Dec. 8, 2009, which claims priority under 35 U.S.C. 119(a) to Patent Application No. 10-2008-0126415 filed in the Republic of Korea on Dec. 12, 2008, Patent Application No. 10-2008-0136859 filed in the Republic of Korea on Dec. 30, 2008, Patent Application No. 10-2008-0136840 filed in the Republic of Korea on Dec. 30, 2008, and Patent Application No. 10-2009-0004872 filed in the Republic of Korea on Jan. 21, 2009. All of these applications are hereby expressly incorporated by reference into the present application.

### BACKGROUND

The present invention relates to a medical apparatus, more particularly to a surgical instrument.

In the field of medicine, surgery refers to a procedure in which a medical apparatus is used to make a cut or an incision in or otherwise manipulate a patient's skin, mucosa, or other tissue, to treat a pathological condition. A surgical procedure such as a laparotomy, etc., in which the skin is cut open and an internal organ, etc., is treated, reconstructed, or excised, may entail problems of blood loss, side effects, pain, and scars. Thus, current methods of surgery that involve making an incision in the skin and inserting only a medical apparatus, such as a laparoscope, a surgical instrument, and a microscope, for example, or those that involve the use of surgical robots are currently regarded as popular alternatives.

A set of surgical robots may include a master robot, which is manipulated by the doctor to generate and transmit the necessary signals, and a slave robot, which receives the signals from the master robot to actually apply the manipulation to the patient. The master robot and the slave robot can be arranged in the operating room as an integrated unit or as separate devices.

A slave robot may be equipped with a robot arm to make manipulations for surgery, while an instrument may be mounted on the front end of the robot arm. As illustrated in FIG. 1, a conventional instrument 54 for mounting on a robot arm may include a driving part 108, a shaft 102 extending from the driving part 108, and a forceps-like effector 112 mounted on the far end 106 of the shaft 102 that is to be inserted into the surgical site.

On a bottom surface of this type of conventional instrument 54, a multiple number of drive wheels (not shown) may be joined. Wires connected to different portions of the effector 112 may be respectively pulley-joined with the drive wheels, so that the rotations of the drive wheels may apply tension to the wires, causing the portions of the effector 112 to move and thus grab or cut the surgical site.

Although this surgical instrument may be suitable for procedures such as laparoscopic surgery that involve making multiple incisions, it may not be so suitable for surgical procedures in which only one incision is made. Procedures for single port access (SPA) surgery or microsurgery, for example, include inserting a vision system (a laparoscope, microscope, etc.) and a surgical instrument all through one hole. In current microsurgery procedures, such as for replantation surgery, spine surgery, brain surgery, etc., a microscope and a surgical instrument may be inserted after making just one hole or a slit having a length of 1 to 2 cm, instead of making multiple incisions. A conventional surgical instrument (including those for robot surgery) may not provide a desired level of freedom in such microsurgery or SPA surgery procedures. That is, if several of these surgical instruments are placed through one hole or a small slit, the instruments' housings 108, i.e. the couplers, may obstruct one another, making it very inconvenient to use conventional surgical instruments in these types of surgeries.

Also, according to the related art, it can be difficult for a user to utilize a surgical instrument in a convenient and efficient manner, as the user is unable to bend or unbend the shaft 102 at will.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention is to provide a flexible surgical instrument that can be bent or unbent arbitrarily by the user and can be used in multiple numbers without obstructing one another.

Other technical problems addressed by the present invention will be readily understood from the descriptions that follow.

One aspect of the present invention provides a flexible surgical instrument that has an effector for engaging the surgical site joined to one end and a driving part for operating the effector joined to the other end. This flexible surgical instrument includes: a shaft, which has one end joined with the driving part, extends along a particular lengthwise direction, and includes a bending part that is capable of bending; and a cover part, which holds the bending part of the shaft.

The distance between one end of the shaft and the bending part can be different from the distance between the other end of the shaft and the bending part, and the bending part can be bent by a force applied by a user.

Another aspect of the present invention provides a flexible surgical instrument that has an effector for engaging the surgical site joined to one end and a driving part for operating the effector joined to the other end. This flexible surgical instrument includes: a shaft, which has one end joined with the driving part, extends along a particular lengthwise direction, and has a flexible form; and a cover part, which holds a bent portion of the shaft to maintain the bent angle of the shaft.

The shaft can be made from a flexible material or can be made as a flexible structure. In such cases, the shaft may be a corrugated tube made of metal or synthetic resin.

The cover part can be flexible or rigid, and can be detachable and attachable in relation to the shaft.

The driving part can be a coupler that includes a driving wheel operated by a driving force transferred from a surgical robot arm, to which the flexible surgical instrument is joined.

Still another aspect of the present invention provides a flexible robotic surgical instrument for mounting on the front end of a surgical robot arm that includes an actuator. This flexible robotic surgical instrument includes: a coupler, which includes a driving wheel operated by a driving force transferred from the actuator; a shaft, which has one end joined with the coupler, extends along a particular lengthwise direction, and is capable of bending; a cover part, which holds a bent portion of the shaft to maintain the bent angle of the shaft; and an effector joined to the other end of the shaft for inserting into the body of a surgery patient.

Here, the shaft can include a first bending part having a bendable form, and the cover part can hold the first bending part. The shaft can be made from a flexible material or can be made as a flexible structure.

Here, the shaft can be a corrugated tube made of metal or synthetic resin, and the cover part can include an angle adjusting part for adjusting the bending angle in correspondence with the bent portion of the shaft.

Furthermore, the angle adjusting part can be a stopper or a screw, and the shaft can be rotatable about an axis following the lengthwise direction.

The distance between one end of the shaft and the bent portion can be different from the distance between the other end of the shaft and the bent portion.

The driving wheel can be shaped as a circular disk and can be configured to clutch onto the actuator to receive a driving force transferred from the actuator.

The flexible robotic surgical instrument can also further include a second bending part, which has a bendable form, positioned between the shaft and the effector. A wire can be joined to the driving wheel to apply a tensional force that bends the second bending part in a particular direction.

Yet another aspect of the present invention provides a medical trocar that includes: a tube-shaped cannula; and a trocar housing, which includes a housing hole connected to an opening of the cannula, joined to one end of the cannula. The cannula is flexible, so that a surgical instrument having a bendable shaft may be inserted through the cannula.

The trocar housing can include a drive valve, to which a wire may be joined that applies a tensional force to bend the cannula in a particular direction. The drive valve can be moved by a driving wheel, which in turn may be operated by a driving force transferred from a surgical robot arm to which the surgical instrument is joined. A multiple number of holes can be perforated in the trocar through which to insert a multiple number of instruments.

Additional aspects, features, and advantages, other than those described above, will be obvious from the claims and written description below.

Certain embodiments of the present invention make it possible to conduct surgery using several surgical instruments without having the instruments obstruct one another, and a surgical instrument can be made to have different usage modes according to what length it is set to.

### BRIEF DESCRIPTION OF THE DRAWINGS

FIG. 1 is a perspective view of a surgical instrument according to the related art.

FIG. 2 is a perspective view of a flexible surgical instrument according to an embodiment of the present invention.

FIG. 3 is a perspective view illustrating the bending part of a flexible surgical instrument according to an embodiment of the present invention.

FIG. 4 and FIG. 5 are diagrams illustrating the uses of flexible surgical instruments according to embodiments of the present invention.

FIG. 6 is a perspective view of a flexible surgical instrument according to another embodiment of the present invention.

FIG. 7 is a perspective view illustrating the bending part of a flexible surgical instrument according to another embodiment of the present invention.

FIG. 8 is a perspective view of a flexible surgical instrument according to another embodiment of the present invention.

FIG. 9 is a drawing illustrating a cover part of a flexible surgical instrument according to an embodiment of the present invention.

FIG. 10 is a drawing illustrating a linking structure between a flexible surgical instrument and a medical trocar according to an embodiment of the present invention.

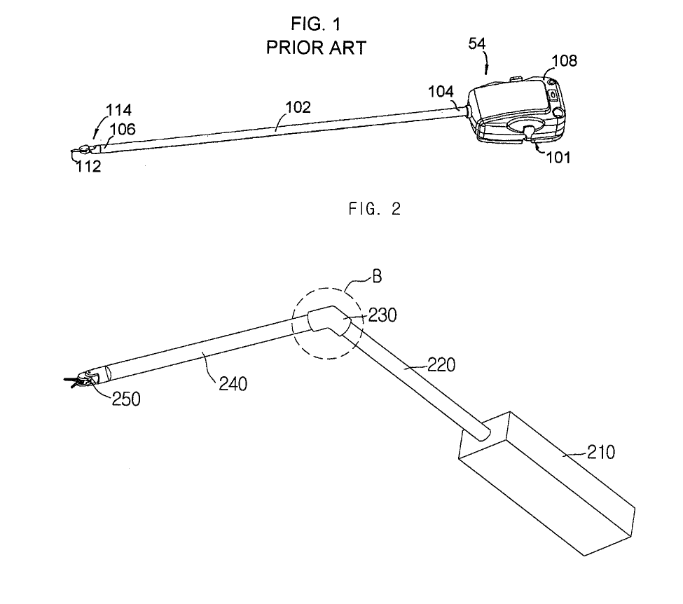

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention.

While terms including ordinal numbers, such as “first” and “second,” etc., may be used to describe various components, such components are not limited to the above terms. The above terms are used only to distinguish one component from another. For example, a first component can be referred to as a second component without departing from the scope of claims of the present invention, and likewise, a second component can be referred to as a first component. If a component is said to be “connected to” or “accessing” another component, it is to be appreciated that the two components can be directly connected to or directly accessing each other but can also include one or more other components in-between.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

FIG. 2 is a perspective view of a flexible surgical instrument according to an embodiment of the present invention. Illustrated in FIG. 2 are a coupler 210, a shaft front part 220, a cover part 230, a shaft rear part 240, and an effector 250.

A feature of this embodiment is that the shaft can be bent by a force applied by the user, so that several surgical instruments may be used during actual surgery without obstructing one another, and the surgical procedures may be facilitated. That is, a first bending part may be provided in a certain position of the shaft, such as the middle position, for example. Then, during surgery, the user may bend this first bending part to a certain angle, and afterwards cover the first bending part with the cover part 230, so that the couplers 210 may not obstruct one another. The first bending part can be held in the cover part 230 to be used during surgery in a bent state.

The cover part 230 can be formed as a detachably attachable structure. For example, the cover part 230 can be shaped as a tube that is bent by a preset angle and can be made of two members that bisect the cross section of the tube. In this case, the user may bend the first bending part to a desired angle, select a cover part 230 that corresponds to the bent angle, and position the cover part 230 to cover the first bending part.

The cover part 230 can be of a flexible or a rigid form. In cases where the cover part 230 is flexible, the user may apply force on the first bending part and bend the shaft to a particular angle while the first bending part is held in the cover part 230. For this purpose, the cover part 230 can be made from a material that is bendable when an amount of force greater than a particular value is applied. In cases where the cover part 230 is rigid, the user may bend the first bending part to a particular angle, and then encase the first bending part with the unbendable cover part 230, so that this shape may be preserved.

According to another embodiment, the whole shaft can be made from a bendable material or made as a bendable structure, instead of having the first bending part in only a particular point of the shaft. In this case, a bent position can be referred to as the first bending part, and in order to maintain this bent shape, the first bending part can be held in the cover part 230.

A flexible surgical instrument according to this embodiment can be used in robotic surgery or in manual surgery. For the former case, the surgical instrument may be mounted on the front end of a surgical robot arm that is equipped with an actuator. Surgery may be conducted as a driving force transferred from the actuator operates a driving wheel (not shown) equipped in the coupler 210, causing the effector 250, which is connected with the driving wheel and is inserted into the body of the surgery patient, to perform a particular maneuver. The driving wheel can be shaped as a circular disk and may clutch onto the actuator to receive the driving force. The number of driving wheels can be determined in correspondence with the number of objects that require control. Details related to the driving wheel are obvious to the person skilled in the field of surgical instruments and thus will be omitted here.

For the latter case, the coupler 210 may be replaced by a particular driving part (not shown), for example an interface (shaped as sticks, buttons, forceps, levers, etc.) that can be directly manipulated by a doctor. Surgery may be performed when the doctor controls the interface, causing the effector 250, which is connected to the interface and inserted into the body of the surgery patient, to perform a particular maneuver. The following descriptions will be provided referring mainly to the former case.

The shaft front part 220 may have one end joined with the coupler 210 and may extend along a first lengthwise direction to join with the first bending part held in the cover part 230. The shaft rear part 240 may have one end joined with the first bending part and may extend along a second lengthwise direction that forms a certain angle with the shaft front part 220.

Here, the first lengthwise direction and the second lengthwise direction are different directions, and the angle formed by the two directions can be adjusted within a range that enables greater utility during actual surgery. Since the shaft front part 220 may extend along the first lengthwise direction and the shaft rear part 240 may extend along the second lengthwise direction, there is a lower risk that the couplers 210 will obstruct one another, when more than one of such surgical instruments are used in surgery. Therefore, the surgical instruments and robotic surgery can be applied even to microsurgery and SPA surgery procedures. In particular, if a surgical instrument structured as above, i.e. folded and extending along a first direction and a second direction, is joined with a robot arm, a greater level of freedom can be provided in terms of the direction in which the robot arm is installed and the direction in which the surgical instrument is extended, etc. The user may thus utilize the surgical instruments in a manner similar to using one's own arms, making it possible to conceive new surgical techniques.

In this embodiment, a wire can be used for connecting the driving wheel with the effector 250. That is, when the driving wheel is rotated, the movements of the effector 250 can be controlled as the wire joined with the driving wheel is contracted or relaxed. The number of driving wheels can be determined in correspondence with the structure for controlling the movements of the effector 250 and the number of wires used.

The effector 250 may be joined to the other end of the shaft rear part 240 and may be inserted into the body of the surgery patient. The effector 250 is the member that engages the surgical site during actual surgery. The effector 250 of the surgical instrument may include a pair of jaws, which may perform a gripping or cutting movement. Also, the effector 250 can be formed such that the whole of the effector 250 is able to rotate in linkage with the rotation of the shaft rear part 240.

In this case, the driving wheels of the driving part can be pulley-joined with the pair of jaws. Various methods can be used for joining the driving wheels with the pair of jaws, such as joining a set of wires to each of the jaws or joining a set of wires to the pair of jaws, for example. Referring to the latter case, as the driving wheels are rotated, the driving forces may be transferred by way of the wires, so that the pair of jaws may perform a gripping or cutting movement. In moving the pair of jaws using a set of pulley-wires, the pair of jaws may be connected by gears, etc., and the pulley-wires can be joined to one of the pair of jaws or to a portion where the pair of jaws are joined, to transfer the driving forces. Of course, various other mechanisms can be applied in which a set of pulleys are used that enable the pair of jaws to perform a gripping movement.

The shaft can be made to rotate about the first lengthwise direction, in which the shaft front part 220 is extended. In this case, the whole of the shaft rear part 240 can also rotate in correspondence with the rotation of the shaft front part 220, while extending in the second lengthwise direction.

Also, according to another embodiment, the shaft front part 220 and the shaft rear part 240 can be joined to each other in such a way that the shaft rear part 240 is rotatable at the first bending part about an axis following the second lengthwise direction described above. For example, the shaft front part 220 and the shaft rear part 240 can be bearing-joined with each other. Here, a bearing-joint refers to a joint that enables smooth rotational motion by reducing friction between the shaft front part 220 and the shaft rear part 240.

To enable this rotation of the shaft rear part 240, a separate wire can be used with one end joined with the driving wheel and the other end joined with the shaft rear part 240. Various methods can be used by which this wire joins the shaft rear part 240, such as winding the wire around the shaft rear part 240, or affixing the wire to a certain point on the shaft rear part 240, for example. Of course, various other mechanisms for rotating the shaft rear part 240 using a wire can be applied to this embodiment.

As described above, the shaft front part 220 and the shaft rear part 240 may be joined in such a way that the shaft rear part 240 is able to rotate about an axis following the second lengthwise direction. In this specification, this joining method will be referred to as a “bearing-joint.” Here, a bearing-joint not only includes linking structures such as a ball bearing, roller bearing, and plate bearing, but also encompasses various other linking structures, such as a screw-joint along an axis following the second lengthwise direction, and a linking structure that surrounds the perimeter of the shaft rear part 240 and uses a linking member that is held in an indentation formed in the perimeter. This bearing joint may have a rotatable structure, to allow the shaft rear part 240 to rotate while extending in the second lengthwise direction, and obviously, various other bearing-joints can be applied to this embodiment.

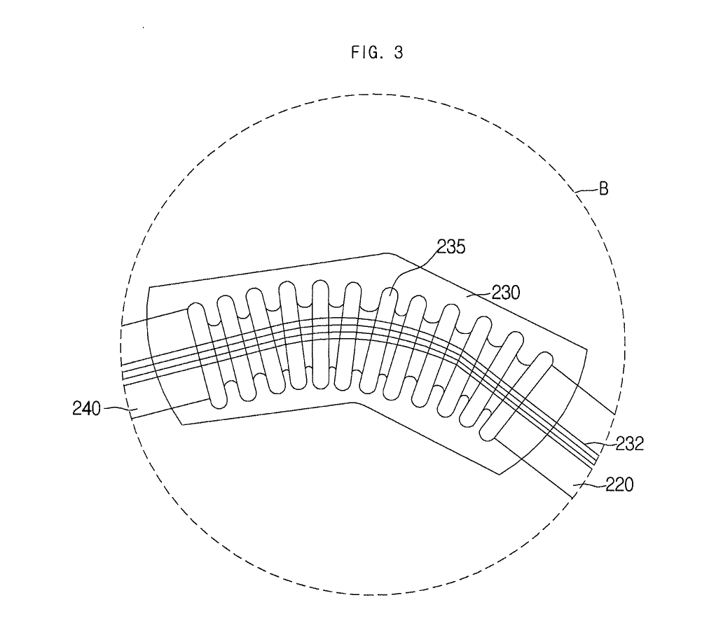

FIG. 3 is a perspective view illustrating the bending part of a flexible surgical instrument according to an embodiment of the present invention. In FIG. 3, which is a magnified view of area B, there are illustrated a shaft front part 220, a cover part 230, first wires 232, a first bending part 235, and a shaft rear part 240.

A first wire 232 may join the driving wheel with the effector 250 such that the effector 250 can be moved by the operation of the driving wheel. The first wire 232 may be pulley-joined to the driving wheel, to be moved in one direction in correspondence with the rotation of the driving wheel, where the effector 250 may perform a particular action in correspondence with this movement. For joining the first wire 232 to the driving wheel and the effector 250, a hole can be formed in the first bending part 235 along the direction in which the first bending part 235 is extended, and the first wire 232 can extend through this hole.

According to another embodiment, the first wire 232 may have one end joined to a portion of the driving wheel and the other end joined to a portion of the effector 250. The rotational movement of the driving wheel may cause the first wire 232 to undergo a contracting or relaxing motion, and in correspondence to this motion, the effector 250 may perform a particular operation, such as a gripping operation or a cutting operation.

The first bending part 235 may be formed as a bendable structure or from a bendable material. For example, the first bending part 235 can be a structure that includes a multiple number of separate articulated parts and is bent when a certain amount of force is applied in a particular direction. Also, the first bending part 235 can be made from a material high in plasticity, such as a synthetic resin tube. Furthermore, the whole shaft can be made as a bendable structure or made from a bendable material, with a cover part 230 used for encasing the bent location and maintaining its shape.

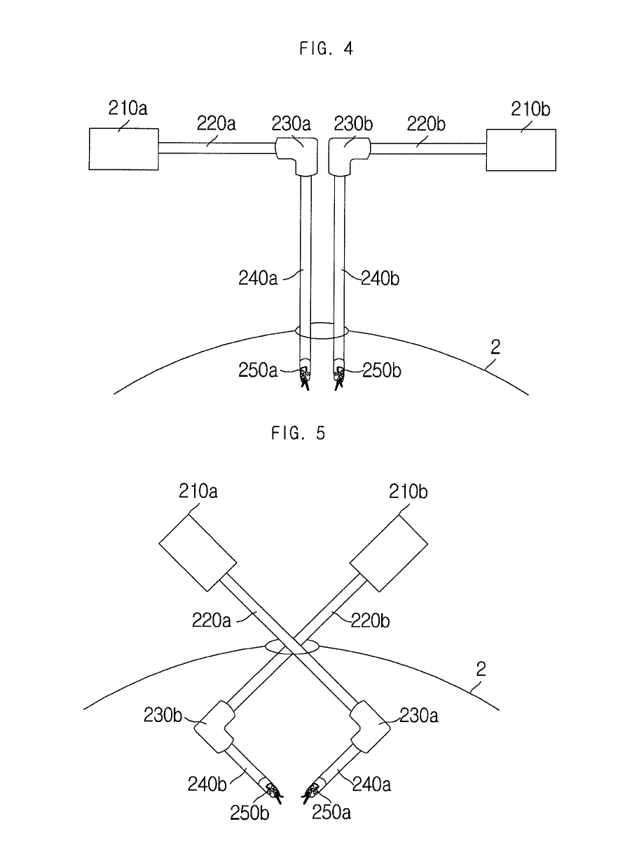

FIG. 4 and FIG. 5 are diagrams illustrating the uses of flexible surgical instruments according to embodiments of the present invention. Illustrated in FIG. 4 and FIG. 5 are couplers 210a, 210b, shaft front parts 220a, 220b, cover parts 230a, 230b, shaft rear parts 240a, 240b, effectors 250a, 250b, and a surgery patient 2.

In the example shown in FIG. 4, two flexible surgical instruments according to this embodiment are inserted through one hole formed in the skin of the surgery patient 2, and the cover parts 230a, 230b are not inserted through the hole but are positioned outside the skin of the surgery patient 2. As the shaft front parts 220a, 220b, which join with the shaft rear parts 240a, 240b, may extend in different directions, the couplers 210a, 210b may not obstruct each other. Here, the lengths of the shaft rear parts 240a, 240b can be greater than the lengths of the shaft front parts 220a, 220b. A laparoscope can additionally be inserted when conducting laparoscopic surgery, and a microscope can additionally be inserted when conducting microsurgery, but vision systems such as the laparoscope or microscope have been omitted from the drawings for more convenience.

In the example shown in FIG. 5, two flexible surgical instruments according to this embodiment are inserted through one hole formed in the skin of the surgery patient 2, and the cover parts 230a, 230b are inserted through the hole, to be positioned inside the skin of the surgery patient 2. To conduct surgery more smoothly for this situation, the lengths of the shaft rear parts 240a, 240b can be shorter than the lengths of the shaft front parts 220a, 220b. For example, when conducting SPA surgery, the effectors 250a, 250b can be moved towards the surgical site more easily and more efficiently, if the second shaft rear parts 240a, 240b are shorter than the shaft front parts 220a, 220b as in FIG. 5.

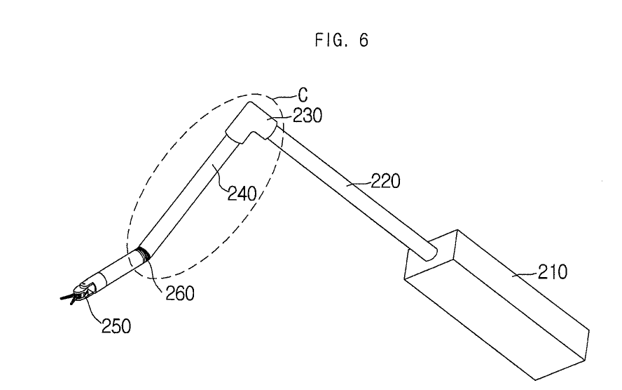

FIG. 6 is a perspective view of a flexible surgical instrument according to another embodiment of the present invention. Illustrated in FIG. 6 are a coupler 210, a shaft front part 220, a cover part 230, a shaft rear part 240, an effector 250, and a second bending part 260. The following descriptions will focus mainly on the differences from the previously described embodiment.

The second bending part 260 may be positioned between the shaft rear part 240 and the effector 250 and may have a bendable structure. Here, to state that the second bending part 260 may be positioned between the shaft rear part 240 and the effector 250 is intended to encompass not only those cases where the second bending part 260, i.e. a bendable member, is formed over all of the length between the shaft rear part 240 and the effector 250, but also those cases where the second bending part 260 is included at one end of the shaft rear part 240 and the effector 250 is joined to the far end after a particular length extending from the second bending part 260, as illustrated in the drawing.

The second bending part 260 may form a particular angle with the second lengthwise direction in which the shaft rear part 240 is extended, and may be formed as a bendable structure or from a bendable material. Similar to the first bending part 235 described above, the second bending part 260 can be a structure that includes a multiple number of separate articulated parts and is bent when a certain amount of force is applied in a particular direction or can be made from a material high in plasticity, such as a synthetic resin tube.

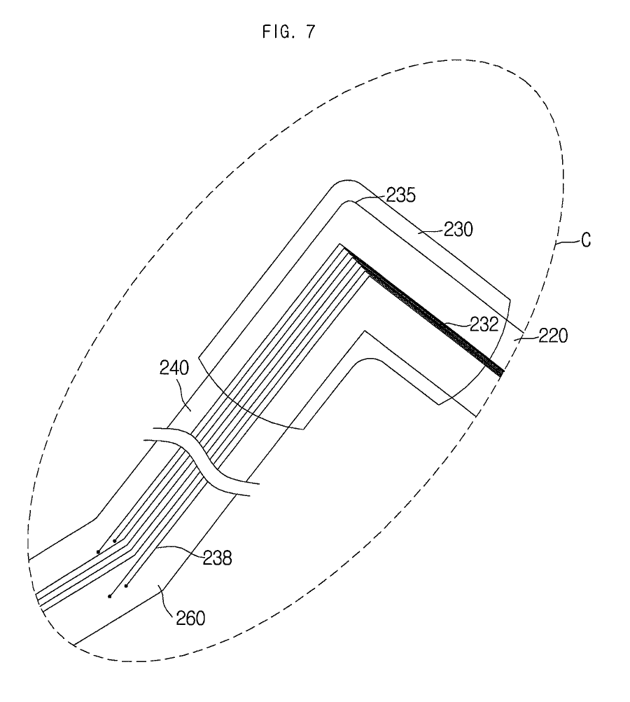

The second bending part 260 may be controlled by the operation of driving wheels, and for this purpose, the second bending part 260 and the driving wheels can be connected by wires. Referring to FIG. 7, which is a magnified view of area C, second wires 238 may connect the driving wheels with the second bending part 260, whereby the movement of the second bending part 260 can be controlled by the manipulation of the driving wheels. The second wires 238 may each have one end attached to one of four portions, respectively, within the second bending part 260, for example in intervals of 90 degrees. The other ends of the second wires 238 may be joined to the driving wheels, and the rotational movements of the driving wheels may contract or relax the second wires 238 to adjust the tensional forces applied, so that the angle and direction in which the second bending part 260 is bent may be determined accordingly. To implement such movements, additional driving wheels can be provided for manipulating the second bending part 260. Of course, various other mechanisms for bending the second bending part 260 using the second wires 238 can be applied to this embodiment.

Providing the surgical instrument with the second bending part 260 can increase the degree of freedom in controlling movements, so that the surgery may be conducted with greater convenience. That is, if second bending parts 260 are included in the examples of FIG. 4 and FIG. 5, the effectors 250a, 250b may be positioned in the surgical site more conveniently and more efficiently.

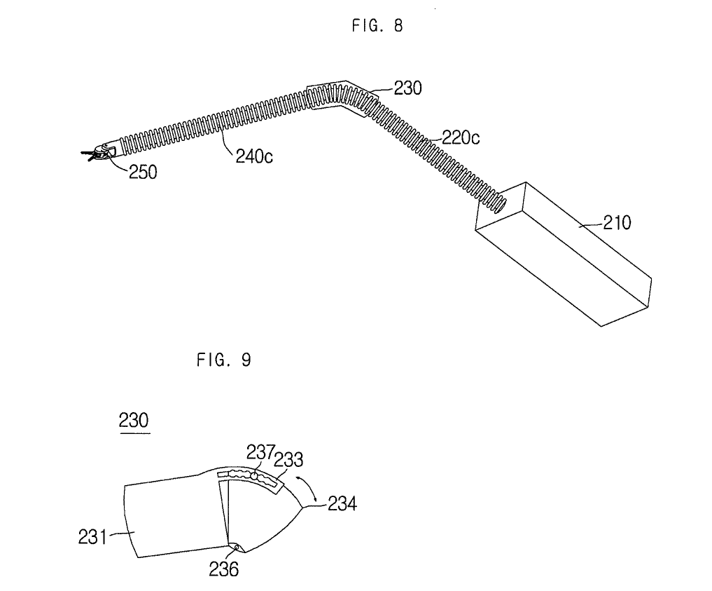

FIG. 8 is a perspective view of a flexible surgical instrument according to another embodiment of the present invention. Illustrated in FIG. 8 are a coupler 210, a shaft front part 220c, a cover part 230, a shaft rear part 240c, and an effector 250. The following descriptions will focus mainly on the differences from the previously described embodiments.

A feature of this embodiment is that the whole shaft 220c, 240c is implemented in a flexible form. This may be achieved by forming the shaft 220c, 240c from a material which is itself bendable or by forming the shaft 220c, 240c as a bendable structure. The shaft 220c, 240c may bend when the user applies an amount of force greater than a threshold value, and after it is bent, may bend or unbend to another angle when a force greater than the threshold value is applied again. Here, the threshold amount of force can be set such that the flexible surgical instrument according to this embodiment is not randomly unbent or bent in another direction during use in surgery.

For example, the shaft 220c, 240c can be a corrugated tube capable of bending. Here, the corrugated tube can be made from a common synthetic resin or metal, while a laminate made of synthetic resin may be applied on the exterior.

The cover part 230 may serve to hold the bent portion after the user bends the shaft 220c, 240c and to maintain the bent angle of the shaft 220c, 240c. To this end, the cover part 230 can have a form that remains secured in an angled state. In this case, several types of cover parts 230 can be prepared, each bent at a different angle. After determining the angle by which the shaft 220c, 240c is to be bent when conducting surgery, the user may select the cover part 230 corresponding to this angle and position the cover part 230 at the bent portion of the shaft 220c, 240c, so that the shaft 220c, 240c may maintain its bent angle.

According to another embodiment, the cover part 230 itself can also be made flexible. In this case, the cover part 230 can be stiffer and more resistant to bending, compared to the shaft 220c, 240c. That is, in order that the cover part 230 may serve to maintain the bent state of the shaft 220c, 240c, the threshold force described above can be greater for the cover part 230 compared to the shaft 220c, 240c.

FIG. 9 is a drawing illustrating a cover part of a flexible surgical instrument according to an embodiment of the present invention. Illustrated in FIG. 9 are a first cover part 231, a stopper 233, a second cover part 234, a rotational axis 236, and a fastening part 237.

According to this embodiment, a cover part 230 is provided which can be varied in its bending angle while maintaining a rigid state. This cover part 230 can be adjusted in correspondence to the angle of the shaft 220, 240 in a flexible surgical instrument according to this embodiment.

The cover part 230 may have a first cover part 231 extending towards the shaft front part 220c and a second cover part 234 extending towards the shaft rear part 240c. The first cover part 231 and second cover part 234 can be hinge-joined about a rotational axis 236, to be capable of rotational movement. A stopper 233 can, in linkage with a fastening part 237, adjust the joint angle between the first cover part 231 and the second cover part 234. That is, the stopper 233 can be joined to the first cover part 231 and can include a multiple number of detent curbs formed along a particular circumference centering about the rotational axis 236. The fastening part 237 can be joined to one of the detent curbs, to secure the second cover part 234 in a rotated position about the rotational axis 236. For this purpose, the fastening part 237 can be formed as a protrusion in a particular position of the second cover part 234.

Using this structure, the cover part 230 can be secured while maintaining a particular angle between the first cover part 231 and the second cover part 234. Also, the fastening part 237 can be screw-joined with the second cover part 234. When the second cover part 234 is to be rotated in relation to the first cover part 231, for example, the screw-joint of the fastening part 237 can be unscrewed, to rotate the second cover part 234, and then tightened again, to secure the second cover part 234.

While the description above has been set forth with reference to an example in which the stopper 233 is formed in the first cover part 231 and the fastening part 237 is formed on the second cover part 234, it is obvious that that the stopper 233 can be formed in the second cover part 234 and the fastening part 237 can be formed on the first cover part 231.

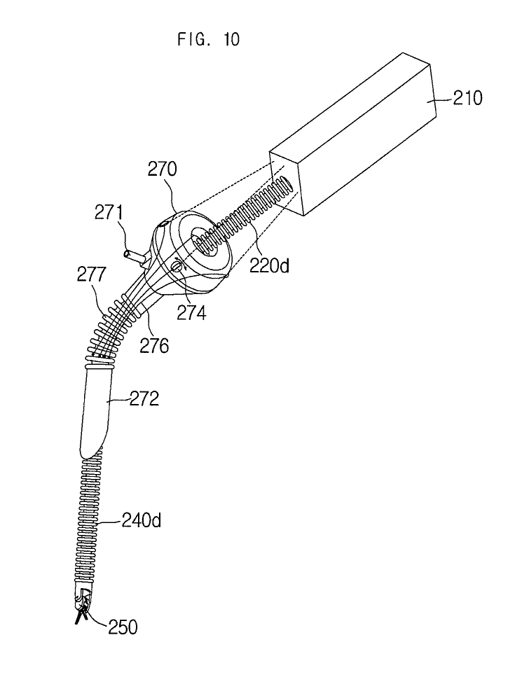

FIG. 10 is a drawing illustrating a linking structure between a flexible surgical instrument and a medical trocar according to an embodiment of the present invention. Illustrated in FIG. 10 are a coupler 210, a shaft front part 220d, a medical trocar, a shaft rear part 240d, and an effector 250. The medical trocar can include a trocar housing 270, a vent tube 271, a cannula 272, drive valves 274, third wires 276, and a third bending part 277. The following descriptions will focus mainly on the differences from the previously described embodiments.

A medical trocar is a medical tool typically used to access the abdominal cavity. During surgery, a medical tool such as a laparoscope and a surgical instrument may be inserted using a medical trocar. In order to insert a flexible surgical instrument such as those described above, a medical trocar according to this embodiment can be made with a flexible form.

The cannula 272, which is to be inserted through the skin of the patient, can include a third bending part 277 that can be bent at a particular position. The third bending part 277 can be implemented by a particular material or structure as described above. Also, according to another embodiment, the whole of the cannula 272 can have a flexible form. Since this structure can be implemented in a manner similar to the shaft of the flexible surgical instrument described above, details on this matter will be omitted. As described above, the threshold force required for bending the cannula 272 can be greater than the threshold force for bending the flexible surgical instrument, whereby the cannula 272 can maintain the bent angle of the flexible surgical instrument.

Gases within the body can be exhausted to a pre-arranged location (e.g. a vacuum suction tube or an air vent of the operating room) through the cannula 272, as well as a vent tube 271 and a vacuum connection tube (not shown), which may be prepared additionally.

The drive valves 274 can be provided to adjust the angle by which the cannula 272 is bent. That is, the drive valves 274 and certain points on the cannula 272 may be connected by third wires 276, where the rotation or movement of the drive valves 274 may adjust the tensional forces applied on the third wires 276 and thus bend the cannula 272 in a particular direction.

That is, the third wires 276 may each have one end attached to one of four portions, respectively, within the cannula 272, for example in intervals of 90 degrees. The other ends of the third wires 276 may be joined to the drive valve 274, and the rotational movements of the drive valve 274 may contract or relax the third wires 276 to adjust the tensional forces applied, so that the angle and direction in which the cannula 272 is bent may be determined accordingly. Of course, various other mechanisms for bending the cannula 272 using the third wires 276 can be applied to this embodiment.

According to another embodiment, the drive valves 274 can be connected by wires to driving wheels of the coupler 210. That is, the manipulation of the actuator of the robot arm can move the driving wheels of the coupler 210, and the drive valves 274 can be controlled correspondingly to bend the cannula 272 in a particular direction. This embodiment provides the advantage that a user may bend the medical trocar at will using a master robot.

While FIG. 10 illustrates an example in which the medical trocar includes one passageway through which to insert a medical tool, the present invention is not thus limited. A medical trocar according to another embodiment can include multiple passageways, for example with several holes perforated for single port surgery.

Other details related to the flexible surgical instrument according to an embodiment of the present invention or related to the surgical robot which the instrument may operate in linkage with, including, for example, detailed mechanical designs, common platform technology, such as the embedded system, O/S, etc., interface standardization technology, such as the communication protocol, I/O interface, etc., and component standardization technology, such as for actuators, batteries, cameras, sensors, etc., are obvious to those of ordinary skill in the field of art to which the present invention belongs and thus will be omitted here.

While the flexible surgical instrument according to an embodiment of the present invention has been described above with reference to certain examples regarding the number and functions of the shafts, the present invention is not thus limited. Other compositions, in which the shaft is divided into smaller segments, or in which the operation method does not utilize wires, for example, can be encompassed by the scope of claims of the present invention if the overall actions and effects are substantially the same.

While the present invention has been described with reference to particular embodiments, it will be appreciated by those skilled in the art that various changes and modifications can be made without departing from the spirit and scope of the present invention, as defined by the claims appended below.

What is claimed is:

1. A flexible surgical instrument having an effector joined to one end thereof and a driving part joined to the other end thereof, the effector configured to engage a surgical site, the driving part configured to operate the effector, the flexible surgical instrument comprising:
a shaft having one end thereof joined with the driving part and extending along a particular lengthwise direction, the shaft comprising a bending part having a bendable form; and
an angle maintaining part being shaped as a tube that is bent by a preset angle, the angle maintaining part holding the bending part therein and maintaining an angled state.

2. The flexible surgical instrument according to claim 1, wherein a distance between one end of the shaft and the bending part is different from a distance between the other end of the shaft and the bending part.

3. The flexible surgical instrument according to claim 1, wherein the bending part is capable of being bent by a force applied by a user.

4. A flexible surgical instrument having an effector joined to one end thereof and a driving part joined to the other end thereof, the effector configured to engage a surgical site, the driving part configured to operate the effector, the flexible surgical instrument comprising:
a shaft having one end thereof joined with the driving part and extending along a particular lengthwise direction, the shaft having a flexible form; and
an angle maintaining part being shaped as a tube that is bent by a preset angle, the angle maintaining part configured to hold a bent portion of the shaft therein and maintain a bent angle of the shaft.

5. The flexible surgical instrument according to claim 4, wherein the shaft is made from a flexible material or is made as a flexible structure.

6. The flexible surgical instrument according to claim 5, wherein the shaft is a corrugated tube made of metal or synthetic resin.

7. The flexible surgical instrument according to claim 4, wherein the driving part is a coupler comprising a driving wheel, the driving wheel configured to operate by a driving force transferred from a surgical robot arm to which the flexible surgical instrument is joined.

8. A flexible robotic surgical instrument for mounting on a front end of a surgical robot arm, the surgical robot arm including an actuator, the flexible robotic surgical instrument comprising:
a coupler comprising a driving wheel, the driving wheel configured to operate by a driving force transferred from the actuator;
a shaft having one end thereof joined with the coupler and extending along a particular lengthwise direction, the shaft having a bendable bending part;
an angle maintaining part being shaped as a tube that is bent by a preset angle, the angle maintaining part configured to hold the bending part therein and maintain an angled state of the bending part; and
an effector for inserting into a body of a surgery patient, the effector joined to the other end of the shaft.

9. The flexible robotic surgical instrument according to claim 8, wherein the shaft is made from a flexible material or is made as a flexible structure.

10. The flexible robotic surgical instrument according to claim 9, wherein the shaft is a corrugated tube made of metal or synthetic resin.

11. The flexible robotic surgical instrument according to claim 8, wherein the angle maintaining part comprises an angle adjusting part capable of adjusting a bending angle of the bending part.

12. The flexible robotic surgical instrument according to claim 11, wherein the angle adjusting part is a stopper or a screw.

13. The flexible robotic surgical instrument according to claim 8, wherein the shaft is rotatable about an axis following the lengthwise direction.

14. The flexible robotic surgical instrument according to claim 8, wherein a distance between one end of the shaft and the bent portion is different from a distance between the other end of the shaft and the bent portion.

15. The flexible robotic surgical instrument according to claim 8, wherein the driving wheel is shaped as a circular disk and is configured to clutch onto the actuator to receive a driving force transferred therefrom.

16. The flexible robotic surgical instrument according to claim 8, further comprising a second bending part having a bendable form, the second bending part positioned between the shaft and the effector.

17. The flexible robotic surgical instrument according to claim 16, wherein the driving wheel has a wire joined thereto, the wire configured to apply a tensional force such that the second bending part is bent in a particular direction.

18. A medical trocar used in surgery using surgical instruments having bendable shaft, the trocar comprising:
a trocar housing having a housing hole through which the shaft is inserted; and
a tube-shaped cannula joined to the trocar housing, through which the shaft is inserted,
wherein the cannula is bendable by a preset angle so as to hold a bent portion of the shaft therein and maintain a bent angle of the shaft.

19. The medical trocar according to claim 18, wherein a wire is joined to the cannula, the bent angle of the cannula is adjusted by a tensional force of the wire, and a drive valve to apply a tensional force to the wire is installed in the trocar housing.

20. The medical trocar according to claim 19, wherein a driving wheel is installed in a surgical robot arm on which the surgical instrument is mounted, and the drive valve is operated by a driving force transferred from the driving wheel.
